(19) United States
(12) Patent Application Publication
(10) Pub. No.: US 2024/0245595 A1
(43) Pub. Date: Jul. 25, 2024

(54) PLACENTA-MIMIC COSMETIC COMPOSITION

(71) Applicant: LG HOUSEHOLD & HEALTH CARE LTD., Seoul (KR)

(72) Inventors: Yun Hee CHANG, Seoul (KR); Mu Hyun JIN, Seoul (KR)

(73) Assignee: LG HOUSEHOLD & HEALTH CARE LTD., Seoul (KR)

(21) Appl. No.: 18/566,247

(22) PCT Filed: Jun. 2, 2022
(86) PCT No.: PCT/KR2022/007822

§ 371 Date: Dec. 1, 2023

(30) Foreign Application Priority Data
Jun. 2, 2021 (KR) 10-2021-0071411

(51) Int. Cl. A61K 8/60; A61K 8/44; A61K 8/49; A61K 8/64; A61K 8/67; A61Q 19/02; A61Q 19/08

(52) U.S. Cl. CPC A61K 8/606; A61K 8/44; A61K 8/442; A61K 8/445; A61K 8/4913; A61K 8/492; A61K 8/494; A61K 8/4946; A61K 8/64; A61K 8/673; A61K 8/675; A61Q 19/02; A61Q 19/08

(57) ABSTRACT

The present invention provides a placenta-mimic cosmetic composition which shows excellent skin improvement effects such as whitening, elasticity improvement, wrinkle improvement, moisturizing, or skin soothing by, instead of directly using the placenta containing ingredients that have legal, ethical, and safety issues, adding HEPES to PDRN, which is a major component of the placenta, and adding peptides, amino acids, or vitamins to same to recombine.

### TECHNICAL FIELD

The present invention relates to a placenta-mimic cosmetic composition that shows excellent skin improvement effects such as whitening, elasticity improvement, wrinkle improvement, moisturizing, or skin soothing by, instead of directly using the placenta containing ingredients that have legal, ethical, and safety issues, adding HEPES to PDRN, which is a major component of the placenta, and adding peptides, amino acids, or vitamins to the same to recombine.

### BACKGROUND ART

Skin is a tissue that covers the outermost part of the body, and always is in contact with the external environment and protects the human body from harmful factors. In addition, since the skin is a visually recognized area, it also determines how beautiful the body is. As signs of aging such as wrinkles, pigmentation, and inflammation accumulate on the skin, its aesthetic value decreases, and thus, there has always been a consumer need for solutions that can prevent or improve these signs.

The placenta is an organ that develops in the uterus of pregnant mammals. The placenta connects the fetus and the mother's uterine wall and is in charge of functions such as supply of nutrients, gas exchange, and excretion of waste products, enabling the fetus to survive and grow within the mother's body and playing a role in protecting the fetus. Such a placenta includes essential amino acids, nucleic acid components such as DNA and RNA, cytokines, growth factors, and the like, and is known to have effective effects such as whitening, wrinkle improvement, immunity enhancement, fatigue recovery, and the like. In herbal medicine, the placenta is called Jahageo and has been used to treat cases such as lack of Qi and blood and thinness due to illness, cases of blemishes on the face and darkened skin due to consumptive diseases, and the like.

Accordingly, prior literature such as “Cosmetic composition using porcine placenta, human placenta extract or hydrolyzed extract (Korean Patent Laid-Open Publication No. 10-2009-000244)”, and “Cosmetic composition reconstituted with human placenta-derived growth factors and cytokines (Korean Registered Patent No. 10-1289062)” is known. However, when the placenta is included in cosmetics, it is undesirable from a safety perspective because it may include components that cause viral infection or immune rejection. Moreover, since the placenta is an animal raw material, consumers may feel aversion to cosmetics that include it. In addition, growth factors and cytokines are components that currently cannot be used in the field of cosmetics. That is, the related art has limitations in implementing cosmetics that fully show the skin improvement effects of the placenta.

Therefore, there is a need for technology that can solve the safety, ethical, and legal issues of the placenta while maintaining and developing the excellent skin improvement efficacy of the placenta.

### RELATED ART LITERATURE

### Patent Documents

(Patent Document 001) Korean Patent Laid-Open Publication No. 10-2009-0002444
(Patent Document 002) Korean Registered Patent No. 10-1289062

### DISCLOSURE

### Technical Problem

The present invention is directed to providing a placenta-mimic cosmetic composition having safety and effectiveness.

The present invention is also directed to providing a use of a placenta-mimic composition for manufacturing cosmetics, pharmaceutical preparations or food for skin whitening.

The present invention is also directed to providing a use of a placenta-mimic composition for manufacturing cosmetics, pharmaceutical preparations or food for improving skin elasticity.

The present invention is also directed to providing a use of a placenta-mimic composition for manufacturing cosmetics, pharmaceutical preparations or food for improving skin wrinkles.

The present invention is also directed to providing a use of a placenta-mimic composition for manufacturing cosmetics, pharmaceutical preparations or food for skin moisturizing.

The present invention is also directed to providing a use of a placenta-mimic composition for manufacturing cosmetics, pharmaceutical preparations or food for skin soothing.

### Technical Solution

As a means for solving the above problems, one aspect of the present invention provides a placenta-mimic composition including, as an active ingredient, Polydeoxyribonucleotide (PDRN) and Hydroxyethylpiperazine Ethane Sulfonic Acid (HEPES) which are recombined by adding peptides, amino acids, or vitamins.

To achieve the above objective, the present invention provides a method for improving skin such as whitening, improving elasticity, reducing wrinkles, moisturizing, or soothing the skin, which includes steps by applying a placenta-mimic composition to the skin.

To achieve the above objective, the present invention provides a method for improving skin such as whitening, improving elasticity, reducing wrinkles, moisturizing, or soothing the skin, which includes steps by administering a placenta-mimic composition to a subject.

To achieve the above objective, the present invention provides a use of a placenta-mimic composition including, as an active ingredient, Polydeoxyribonucleotide (PDRN) and Hydroxyethylpiperazine Ethane Sulfonic Acid (HEPES) which are recombined by adding peptides, amino acids, or vitamins, for manufacturing the placenta-mimic composition that shows excellent skin improvement effects such as whitening, elasticity improvement, wrinkle improvement, moisturizing, or skin soothing.

Hereinafter, the placenta-mimic composition according to the present invention will be described in more detail.

In the present invention, growth factors, hormones, and cytokines with legal, ethical, and safety issues are excluded.

In the related art, a cosmetic composition including a placenta extract powder hydrolyzed with a proteolytic enzyme as an active ingredient (Korean Patent Laid-Open Publication No. 10-2009-0002444) and a cosmetic composition including growth factors and cytokines extracted from the placenta as active ingredients (Korean Registered Patent No. 10-1289062) are disclosed. However, the above related arts are aimed at extracting components which have not had their safety confirmed and are legally regulated, such as growth-promoting factors, cytokines, epithelial cell-promoting factors, and the like from the placenta or optimizing a mixing ratio for reconstituting the composition to maximize the efficacy of the components. In addition, there is a difference in purpose because there is no description of safety verification for cosmetic compositions containing the aforementioned ingredients. Furthermore, in terms of measuring skin improvement effects, the prior arts suggest only subjective results of wrinkle improvement by visual inspection, rather than through quantitative analysis using specialized equipment to assess the efficacy of active ingredients for skin improvement, such as skin whitening, elasticity, moisturizing, or soothing anti-inflammatory effects. Therefore, Objective results based on above standardized testing methods for are not presented in prior arts, making it difficult to assess the extent of improvement.

On the other hand, specific embodiments of present invention suggest preparation of a placenta-mimic composition, in vitro and in vivo efficacy tests by using standard test methods or specialized analysis equipment. Through the embodiments of present invention, it was confirmed that the placenta-mimic composition, as an active ingredient, could significantly improve each skin conditions and maximize the efficacy of whitening, elasticity, wrinkle, moisturizing, or skin soothing.

In a specific embodiment of the present invention, further improved effects may be achieved by recombining PDRN and HEPES by adding peptides, amino acids, and vitamins. Specifically, in an in vitro efficacy test, it was confirmed that the skin improvement effect of inhibiting melanin production, promoting collagen biosynthesis, promoting hyaluronic acid biosynthesis, or inhibiting NO production was improved in Example 2 (including PDRN, HEPES, peptides, amino acids, and vitamins) compared to Example 1 (including PDRN, and HEPES). In addition, in an in vivo efficacy test, it was confirmed that the skin wrinkle improvement, elasticity improvement, whitening, or moisturizing effect was improved in Examples 3 and 4 (cosmetic composition including Example 2) compared to Comparative Examples 6 and 7 (cosmetic composition without Example 2). Furthermore, the cosmetic composition according to the present invention may eliminate the risk of unexpected contamination, impossibility of implementation (legal regulations), and consumer reluctance by recombining PDRN and HEPES with additional peptides, amino acids, and vitamins.

In the present invention, as the peptides, one or more combinations selected from the group consisting of yeast polypeptides, oligopeptide-1, oligopeptide-2, copper tripeptide-1, and acetyl hexapeptide-8 may be used.

In the present invention, as the amino acids, one or more selected from the group consisting of arginine, tryptophan, threonine, lysine, valine, leucine, phenylalanine, histidine, isoleucine, tyrosine, potassium aspartate, sodium glutamate, glutamine, asparagine, proline, alanine, glycine, and serine may be used.

In the present invention, as the vitamins, one or more selected from the group consisting of biotin, folic acid, cyanocobalamin, niacinamide, pantothenic acid, pyridoxine, riboflavin, and thiamine may be used.

One aspect of the present invention provides a cosmetic composition including the placenta-mimic composition as an active ingredient.

In the present invention, when the placenta-mimic composition is applied to a cosmetic composition, whitening, elasticity improvement, wrinkle improvement, moisturizing, or skin soothing efficacy can be exhibited, and specifically, a skin improvement effect may be obtained by inhibiting melanin synthesis, promoting collagen synthesis, promoting hyaluronic acid synthesis, or inhibiting NO production.

In the present invention, the cosmetic composition may be in a form of a general emulsified formulation and solubilized formulation. For example, the cosmetic composition may have a formulation such as a lotion such as softening lotion, nourishing lotion, or the like, an emulsion such as facial lotion, body lotion, or the like, a cream such as nourishing cream, moisture cream, eye cream, or the like, an essence, cosmetic ointment, balm, spray, gel, pack, sunscreen, makeup base, a foundation such as liquid type, solid type, spray type, or the like, powder, a makeup remover such as cleansing cream, cleansing lotion, cleansing oil, or the like, a cleanser such as cleansing foam, soap, body wash, or the like.

In addition, the cosmetic may include, in addition to the composition of the present invention, an adjuvant commonly used in the field of cosmetology, such as a fatty substance, an organic solvent, a solubilizing agent, a thickening agent and a gelling agent, a softening agent, an antioxidant, a suspending agent, a stabilizer, a foaming agent, a fragrance, a surfactant, water, an ionic or nonionic emulsifier, a filler, metal ion sequestering and chelating agents, a preservative, a vitamin, a blocking agent, a wetting agent, an essential oil, a dye, a pigment, a hydrophilic or lipophilic active agent, a lipid vesicle or, any other components commonly used in cosmetics.

The cosmetic formulation may include a relatively high concentration of the composition of the present invention in a case of wash-off type cosmetics such as makeup removers, cleansers, and the like in which active ingredients stay on the skin for a short period of time. On the other hand, in a case of leave-on type cosmetics such as lotions, emulsions, creams, essence, and the like in which active ingredients stay on the skin for a long period of time, it is acceptable to include the composition of the present invention at a low concentration relative to that used in the wash-off type cosmetics. Although not limited thereto, in a specific embodiment of the present invention, the composition of the present invention may include 0.001 to 5 parts by weight of the placenta-mimic composition based on a total weight of the composition, for example, 0.001 to 0.01 parts by weight, 0.001 to 0.1 parts by weight, 0.001 to 1 part by weight, 0.001 to 2.5 parts by weight, 0.01 to 0.1 parts by weight, 0.01 to 1 part by weight, 0.01 to 2.5 parts by weight, 0.01 to 5 parts by weight, 0.1 to 1 part by weight, 0.1 to 2.5 parts by weight, 0.1 to 5 parts by weight, 1 to 2.5 parts by weight, or 1 to 5 parts by weight.

When the placenta-mimic composition according to the present invention is included at less than 0.001 parts by weight of the total composition, a sufficient skin improvement effect may not be expected, and when it is included at more than 5 parts by weight thereof, unwanted reactions such as allergies may occur or problems with skin safety may occur, and thus, the above range is for preventing these outcomes.

Each of the above-mentioned components included in the cosmetic composition according to the present invention may be included in the cosmetic composition of the present invention, preferably, within a range that does not exceed a maximum usage amount specified in the regulations related to “Cosmetic Use Permission” stipulated by the government of each country. For example, it may comply with the scope specified in the “Cosmetic Safety Technical Specifications” stipulated by the Chinese government.

Another aspect of the present invention provides a quasi-drug composition including the placenta-mimic composition as an active ingredient.

When the placenta-mimic composition of the present invention is applied to a quasi-drug composition, whitening, elasticity improvement, wrinkle improvement, moisturizing, or skin soothing efficacy can be exhibited, and specifically, a skin improvement effect may be obtained by inhibiting melanin synthesis, promoting collagen synthesis, promoting hyaluronic acid synthesis, or inhibition of NO production.

The quasi-drug composition of the present invention may further include a pharmaceutically acceptable carrier, excipient, or diluent, if necessary. The pharmaceutically acceptable carrier, excipient, or diluent is not limited as long as it can achieve the effects of the present invention, and may include, for example, a filler, an extender, a binder, a wetting agent, a disintegrant, a surfactant, a lubricant, a sweetener, a flavoring agent, a preservative, and the like.

The carrier, excipient, or diluent of the present invention is commonly used, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propylhydroxybenzoate, talc, magnesium stearate, mineral oil, and the like, without being limited thereto.

The quasi-drug composition may be used in any form suitable for pharmaceutical preparations, including oral formulations such as powders, granules, tablets, capsules, suspensions, emulsions, syrups, aerosols, and the like, external preparations such as ointments, creams, and the like, suppositories, sterile injection solutions, and the like, and may further include dispersants or stabilizers. Although not limited thereto, in a specific embodiment of the present invention, the composition of the present invention may include 0.001 to 5 parts by weight of the placenta-mimic composition based on a total weight of the composition, for example, 0.001 to 0.01 parts by weight, 0.001 to 0.1 parts by weight, 0.001 to 1 part by weight, 0.001 to 2.5 parts by weight, 0.01 to 0.1 parts by weight, 0.01 to 1 part by weight, 0.01 to 2.5 parts by weight, 0.01 to 5 parts by weight, 0.1 to 1 part by weight, 0.1 to 2.5 parts by weight, 0.1 to 5 parts by weight, 1 to 2.5 parts by weight, or 1 to 5 parts by weight.

When the placenta-mimic composition according to the present invention is included at less than 0.001 parts by weight of the total composition, a sufficient skin improvement effect may not be expected, and when it is included at more than 5 parts by weight thereof, unwanted reactions such as allergies may occur or problems with skin safety may occur, and thus, the above range is for preventing these outcomes.

### Advantageous Effects

A placenta-mimic cosmetic composition according to the present invention can show excellent skin improvement effects such as whitening, elasticity improvement, wrinkle improvement, moisturizing, or skin soothing by adding hydroxyethylpiperazine ethane sulfonic acid (HEPES) to polydeoxyribonucleotide (PDRN), which is an active ingredient in the placenta, and adding peptides, amino acids, or vitamins to recombine.

### BEST MODE

Hereinafter, the present invention will be explained in detail with the following examples. However, the following examples only illustrate the present invention, and the content of the present invention is not limited to the following examples.

Specific examples of a placenta-mimic composition according to the present invention are illustrated below.

### Preparation Example 1. Preparation of Examples and Comparative Examples of Placenta-Mimic Composition

In order to evaluate whitening, collagen biosynthesis, moisturizing, and soothing effects of the placenta-mimic composition of the present invention, Examples and Comparative Examples were prepared as shown in Table 1 below.

TABLE 1

| | | Comparative | Comparative | Comparative | | Comparative | Comparative | |
|---|---|---|---|---|---|---|---|---|
| | | Example | Example | Example | Example | Example | Example | Example |
| | | 1 | 2 | 3 | 1 | 4 | 5 | 2 |
| PDRN | | 0.2 | — | — | 0.2 | 0.2 | — | 0.2 |
| HEPES | | — | 0.4 | — | 0.4 | — | 0.4 | 0.4 |
| Peptides | Oligopeptide-1 | — | — | 0.01 | — | 0.01 | 0.01 | 0.01 |
| | Oligopeptide-2 | — | — | 0.01 | — | 0.01 | 0.01 | 0.01 |
| | Copper tripeptide-1 | — | — | 0.01 | — | 0.01 | 0.01 | 0.01 |
| | Acetyl hexapeptide-8 | — | — | 0.01 | — | 0.01 | 0.01 | 0.01 |
| | Yeast polypeptide | — | — | 0.1 | — | 0.1 | 0.1 | 0.1 |
| Amino | Arginine | — | — | 0.3 | — | 0.3 | 0.3 | 0.3 |
| Acids | Tryptophan | — | — | 0.1 | — | 0.1 | 0.1 | 0.1 |
| | Threonine | — | — | 0.05 | — | 0.05 | 0.05 | 0.05 |
| | Lysine | — | — | 0.04 | — | 0.04 | 0.04 | 0.04 |
| | Valine | — | — | 0.02 | — | 0.02 | 0.02 | 0.02 |
| | Leucine | — | — | 0.02 | — | 0.02 | 0.02 | 0.02 |
| | Phenylalanine | — | — | 0.02 | — | 0.02 | 0.02 | 0.02 |
| | Histidine | — | — | 0.01 | — | 0.01 | 0.01 | 0.01 |
| | Isoleucine | — | — | 0.01 | — | 0.01 | 0.01 | 0.01 |
| | Tyrosine | — | — | 0.02 | — | 0.02 | 0.02 | 0.02 |
| | Potassium aspartate | — | — | 0.18 | — | 0.18 | 0.18 | 0.18 |
| | Sodium glutamate | — | — | 0.34 | — | 0.34 | 0.34 | 0.34 |
| | Glutamine | — | — | 0.1 | — | 0.1 | 0.1 | 0.1 |
| | Asparagine | — | — | 0.1 | — | 0.1 | 0.1 | 0.1 |
| | Proline | — | — | 0.1 | — | 0.1 | 0.1 | 0.1 |
| | Alanine | — | — | 0.08 | — | 0.08 | 0.08 | 0.08 |
| | Glycine | — | — | 0.06 | — | 0.06 | 0.06 | 0.06 |
| | Serine | — | — | 0.04 | — | 0.04 | 0.04 | 0.04 |
| Vitamins | Biotin | — | — | 0.1 | — | 0.1 | 0.1 | 0.1 |
| | Folic Acid | — | — | 0.1 | — | 0.1 | 0.1 | 0.1 |
| | Cyanocobalamin | — | — | 0.1 | — | 0.1 | 0.1 | 0.1 |
| | Niacinamide | — | — | 0.1 | — | 0.1 | 0.1 | 0.1 |
| | Pantothenic acid | — | — | 0.1 | — | 0.1 | 0.1 | 0.1 |
| | Pyridoxine | — | — | 0.1 | — | 0.1 | 0.1 | 0.1 |
| | Riboflavin | — | — | 0.1 | — | 0.1 | 0.1 | 0.1 |
| | Thiamine | — | — | 0.1 | — | 0.1 | 0.1 | 0.1 |

| | | | | | | | |
|---|---|---|---|---|---|---|---|
| Butylene glycol | 20 | 20 | 20 | 20 | 20 | 20 | 20 |
| Purified water | up to 100 | up to 100 | up to 100 | up to 100 | up to 100 | up to 100 | up to 100 |

### Experimental Example 1: Whitening Evaluation (Melanin Content Evaluation)

MNT-1 melanoma cells were seeded in a 6-well plate at a concentration of 1-2×10⁵ cells/ml and cultured for 24 hours. Samples including each of Comparative Examples and Examples at concentrations shown in Table 2 below were prepared in DMEM medium, the cultured melanoma cells were treated with the samples, and further cultured for 72 hours. At this time, a DMEM medium to which no other components were added was used as a control group. When the culture was completed, the cells were collected, centrifuged at 13,000 rpm for 1 minute, and the supernatant was removed, and thereafter, lysis was performed by adding 300 μl of 0.5% Triton X-100 solution to a pellet. This was centrifuged again at 13,000 rpm for 3 minutes, and the pellet and supernatant were separately collected. 100 μl of 0.5N NaOH was added to the cell pellet, incubated overnight to dissolve melanin, and thereafter, an absorbance at 450 nm was measured using an ELISA reader. The ability to inhibit melanin synthesis was evaluated as follows.

$$Ability to inhibit melanin synthesis ( \% ) = ( \frac{Melanin content in control group - melanin content in sample}{Melanin content in control group} ) \times 100$$

The results are shown in Table 2 below.

TABLE 2

| | | Ability to inhibit |
|---|---|---|
| | | melanin synthesis |
| | | (relative value |
| | Treatment | compared to |
| | concentration | control group, %) |
| Comparative | 0.01% | — |
| Example 1 | | |
| Comparative | 0.01% | — |
| Example 2 | | |
| Comparative | 0.01% | 0.2 |
| Example 3 | | |
| Example 1 | 0.01% | 6.8 |
| Comparative | 0.01% | 8.4 |
| Example 4 | | |
| Comparative | 0.01% | 0.2 |
| Example 5 | | |
| Example 2 | 0.01% | 25.2 |

—: No effect

As shown in Table 2, Examples 1 and 2 showed an effect of inhibiting melanin synthesis. Comparative Example 1 including only PDRN had no effect at all, whereas it was confirmed that the ability to inhibit melanin synthesis was increased when recombination with HEPES (Example 1) or peptides, amino acids, and vitamins (Comparative Example 4) was made. In particular, Example 2 showed the best ability to inhibit melanin synthesis, indicating that there is a synergistic efficacy in inhibiting melanin synthesis when PDRN is recombined by adding HEPES, peptides, amino acids, and vitamins.

### Experimental Example 2: Elasticity Improvement Evaluation (Collagen Biosynthesis Evaluation)

Skin fibroblasts were seeded in a 48-well plate at a concentration of 2-5×10⁴ cells/ml and cultured for 24 hours. After removing a growth medium, samples including each of the Comparative Examples and Examples at concentrations shown in Table 3 below were prepared in DMEM medium, the cultured skin fibroblasts were treated with the samples, and further cultured for 24 hours. At this time, a DMEM medium to which no other components were added was used as a control group. When culture was completed, the cell culture medium was taken and ELISA assay was performed. The human procollagen 1α1 duoset ELISA kit used in this experiment was a product of R&D Systems, and evaluation was performed according to the experimental protocol provided by the manufacturer. Collagen biosynthesis was evaluated as follows.

$$Collagen biosynthesis ( \% ) = \{ ( \frac{Sample va1ue}{Control group value} ) \times 100 \} - 100$$

The results are shown in Table 3 below.

TABLE 3

| | | Collagen Biosynthesis |
|---|---|---|
| | Treatment | (relative value compared |
| | Concentration | to control group, %) |
| Comparative | 0.01% | 3.4 |
| Example 1 | | |
| Comparative | 0.01% | — |
| Example 2 | | |
| Comparative | 0.01% | 5.2 |
| Example 3 | | |
| Example 1 | 0.01% | 6.8 |
| Comparative | 0.01% | 5.7 |
| Example 4 | | |
| Comparative | 0.01% | 3.6 |
| Example 5 | | |
| Example 2 | 0.01% | 46.7 |

—: No effect

As shown in Table 3, Examples 1 and 2 showed an effect of promoting collagen biosynthesis. Compared to the effect of Comparative Example 1 including only PDRN, it was confirmed that the effect of promoting collagen biosynthesis was increased when recombination with HEPES (Example 1) or peptides, amino acids, and vitamins (Comparative Example 4) was made. In particular, Example 2 showed the best effect in promoting collagen biosynthesis, indicating that there is a synergistic efficacy in promoting collagen biosynthesis when PDRN is recombined by adding HEPES, peptides, amino acids, and vitamins.

### Experimental Example 3: Moisturizing Evaluation (Hyaluronic Acid Biosynthesis Evaluation)

HaCaT, a human keratinocyte, was seeded in a 48-well plate at a concentration of 2-5×10⁴ cells/ml and cultured for 24 hours. After removing a growth medium, samples including each of the Comparative Examples and Examples at the concentrations shown in Table 4 below were prepared in DMEM medium, the cultured HaCaT were treated with the samples, and further cultured for 48 hours. At this time, a DMEM medium to which no other components were added was used as a control group. When the culture was completed, the cell culture medium was taken, and the moisturizing ability was evaluated by comparing hyaluronic acid synthesis with the control group using Quantikine ELISA Hyaluronan from R&D Systems. Hyaluronic acid biosynthesis was evaluated as follows.

$$Hyaluronuc acid biosynthesis ( \% ) = \{ ( \frac{Sample value}{Control group value} ) \times 100 \} - 100$$

The results are shown in Table 4 below.

TABLE 4

| | | Hyaluronic Acid |
|---|---|---|
| | | Biosynthesis (relative |
| | Treatment | value compared to |
| | Concentration | control group, %) |
| Comparative | 0.01% | — |
| Example 1 | | |
| Comparative | 0.01% | — |
| Example 2 | | |
| Comparative | 0.01% | 3.3 |
| Example 3 | | |
| Example 1 | 0.01% | 8.2 |
| Comparative | 0.01% | 1.2 |
| Example 4 | | |
| Comparative | 0.01% | 0.5 |
| Example 5 | | |
| Example 2 | 0.01% | 25.5 |

—: No effect

As shown in Table 4, Examples 1 and 2 showed an effect of promoting hyaluronic acid biosynthesis. Comparative Example 1 including only PDRN had no effect at all, whereas it was confirmed that the promotion of hyaluronic acid biosynthesis was increased when recombination with HEPES (Example 1) or peptides, amino acids, and vitamins (Comparative Example 4) was made. In particular, Example 2 showed the best effect in promoting hyaluronic acid biosynthesis, indicating that there is a synergistic efficacy in promoting hyaluronic acid biosynthesis when PDRN is recombined by adding HEPES, peptides, amino acids, and vitamins.

### Experimental Example 4: Skin Soothing Evaluation (NO Production Inhibition Evaluation)

Raw 264.7 cells, macrophages, were seeded in a 24-well plate at a concentration of 2×10⁴ cells/ml and cultured for 24 hours. After removing a growth medium, samples including each of the Comparative Examples and Examples at concentrations shown in Table 5 below were prepared in RPMI medium and the cultured macrophages were treated with the samples for 30 minutes. At this time, an RPMI medium to which no other components were added was used as a control group. After sample treatment, 1 μg/ml of LPS was further treated and further cultured for another 24 hours. Subsequently, the ability to inhibit NO production was evaluated using an NO quantification kit as follows.

$$Ability to inhibit NO production ( \% ) = \{ 1 - ( \frac{Sample value}{Control group value} ) \} \times 100$$

The results are shown in Table 5 below.

TABLE 5

| | | Inhibition of NO |
|---|---|---|
| | Treatment | Production (Relative |
| | Concen- | value compared to |
| | tration | control group, %) |
| Comparative | 0.1% | 16.2 |
| Example 1 | | |
| Comparative | 0.1% | 15.4 |
| Example 2 | | |
| Comparative | 0.1% | 1.1 |
| Example 3 | | |
| Example 1 | 0.1% | 15.5 |
| Comparative | 0.1% | 15.9 |
| Example 4 | | |
| Comparative | 0.1% | 15.2 |
| Example 5 | | |
| Example 2 | 0.1% | 43.0 |

As shown in Table 5, Examples 1 and 2 showed an effect of inhibiting NO production. Compared to the effect of Comparative Example 1 including only PDRN, it was confirmed that the effect of inhibiting NO production was significantly increased when recombination with HEPES, peptides, amino acids, and vitamins (Example 2) was made. This shows that there is a synergistic efficacy in inhibiting NO production when PDRN is recombined by adding HEPES, peptides, amino acids, and vitamins.

### Preparation Example 2. Preparation of Example and Comparative Example of Cosmetic Compositions Including Placenta-mimic Composition for Skin Wrinkle, Elasticity, Whitening and Moisturizing Human Tests

In order to evaluate the effect of the placenta-mimic composition on human skin, an Example and a Comparative Example were prepared as cream formulations with the compositions shown in Table 6 below and tested (units: weight %).

TABLE 6

| | Comparative | |
|---|---|---|
| | Example 6 | Example 3 |
| Example 2 | 0 | 1 |
| Purified water | up to 100 | up to 100 |
| Glycerin | 8.0 | 8.0 |
| Butylene glycol | 4.0 | 4.0 |
| Hyaluronic | 5.0 | 5.0 |
| acid extract | | |
| Beta-glucan | 7.0 | 7.0 |
| Carbomer | 0.15 | 0.15 |
| Caprylic/Capric | 8.0 | 8.0 |
| triglyceride | | |
| Squalane | 5.0 | 5.0 |
| Cetearyl glucoside | 1.5 | 1.5 |
| Sorbitan stearate | 0.4 | 0.4 |
| Cetearyl alcohol | 2.0 | 2.0 |
| Preservative | Appropriate | Appropriate |
| | amount | amount |
| Pigment | Appropriate | Appropriate |
| | amount | amount |
| Triethanolamine | 0.15 | 0.15 |

### Experimental Example 5. Skin Wrinkles, Elasticity, Whitening, and Moisturizing Human Test Evaluation

After Example 3 and Comparative Example 6 were applied to the right and left sides of the face of 20 women aged 30 to 50 twice a day in the morning and evening for 8 weeks, respectively, skin wrinkles, elasticity, whitening, and moisturizing effects were measured by the following methods. An improvement rate of each effect was evaluated as follows.

$$Improvement rate ( \% ) = \vert \frac{Measured value after use - measured value before use )}{Measured value before use} \vert \times 100$$

First, skin wrinkles were measured once around the eyes using Antera 3D CS (Miravex, Ireland) equipment, and a depth (mm) of a Wrinkle-small analysis mode was used as evaluation data. The measuring device is a device that can measure a surface image of the skin using a light emitting diode (LED light source), extracts data from a 3D shape image of a built-in program, digitizes the skin condition, and utilizes the image. The results are shown in Table 7 below.

TABLE 7

| | Wrinkle depth |
|---|---|
| | improvement |
| | rate around the |
| | eyes (%) |
| Comparative | 2.1 |
| Example 6 | |
| Example 3 | 10.8 |

As shown in the results in Table 7, a group treated with Example 3 was found to have an excellent wrinkle improvement effect by improving skin wrinkles by 510% or more compared to Comparative Example 6.

Next, the skin elasticity improvement effect was measured using a skin elasticity measuring device (Cutometer SEM 575, C+K Electronic Co., Germany), and the result refers to the skin viscoelasticity measured by the skin elasticity measuring device. The results are shown in Table 8 below.

TABLE 8

| | Skin elasticity |
|---|---|
| | improvement |
| | rate (%) |
| Comparative | 4.2 |
| Example 6 | |
| Example 3 | 12.9 |

As shown in the results in Table 8, the group treated with Example 3 was found to have an excellent skin elasticity effect by improving skin elasticity by 300% or more compared to Comparative Example 6.

Subsequently, a degree of skin tone improvement was evaluated for skin whitening using a Facial Stage DM-3 (Moritex, Japan) device. A skin tone improvement rate was determined by skin brightness and color change values based on skin lightness and color measurement values. The results are shown in Table 9 below.

TABLE 9

| | Comparative Example 6 | Example 3 |
|---|---|---|
| Skin tone | Brightness (mean ± | Brightness (mean ± |
| improvement | standard deviation) | standard deviation) |
| rate (%) | 0.5 ± 0.1 | 2.0 ± 0.3 |
| | Color (mean ± standard | Color (mean ± standard |
| | deviation) | deviation) |
| | 0.7 ± 0.4 | 1.8 ± 0.2 |

As shown in the results in Table 9, the group treated with Example 3 was found to have an excellent skin whitening effect by improving brightness by 400% or more and color by 250% or more compared to Comparative Example 6.

Next, for skin moisturizing, specifically, after washing the face with soap, the skin was adapted to constant temperature and humidity conditions (temperature 20±2° C., relative humidity 40±2%). Using a corneometer (CM825), skin surface capacitance was measured three times repeatedly. The results are shown in Table 10 below.

TABLE 10

| | Moisture |
|---|---|
| | improvement |
| | rate (%) |
| Comparative | 7.2 |
| Example 6 | |
| Example 3 | 22.9 |

As shown in the results in Table 10, the group treated with Example 3 was found to have an excellent skin moisturizing effect by increasing moisture by 310% or more compared to Comparative Example 6.

### Preparation Example 3. Preparation of Example and Comparative Example of Cosmetic Compositions Including Placenta-mimic Composition for Skin Soothing Human Test

In order to evaluate the soothing effect of the placenta-mimic composition on human skin, 5% of lactic acid, a skin irritant, was added, and an Example and a Comparative Example were prepared as cream formulations with the compositions shown in Table 11 below and tested (units: weight %).

TABLE 11

| | Comparative | Example |
|---|---|---|
| | Example 7 | 4 |
| Example 2 | 0 | 1 |
| Purified Water | up to 100 | up to 100 |
| Lactic acid | 5 | 5 |
| Glycerin | 8.0 | 8.0 |
| Butylene glycol | 4.0 | 4.0 |
| Hyaluronic acid | 5.0 | 5.0 |
| Beta-glucan | 7.0 | 7.0 |
| Carbomer | 0.15 | 0.15 |
| Caprylic/Capric triglyceride | 8.0 | 8.0 |
| Squalane | 5.0 | 5.0 |
| Cetearyl glucoside | 1.5 | 1.5 |
| Sorbitan stearate | 0.4 | 0.4 |
| Cetearyl alcohol | 2.0 | 2.0 |
| Preservative | Appropriate | Appropriate |
| | amount | amount |
| Pigment | Appropriate | Appropriate |
| | amount | amount |
| Triethanolamine | 0.15 | 0.15 |

### Experimental Example 6. Skin Soothing Human Test Evaluation

The skin soothing effect was confirmed through a human skin patch experiment on 20 healthy adult men and women in their 20 s to 40 s without skin disease or allergy symptoms and no history of hypersensitivity.

After sufficiently wiping the inside of the forearm of the subject with 70% ethanol, it was completely dried, and 30 μg each of Example 4 and Comparative Example 7 according to Preparation Example 3 was applied to the right and left sides. After 24 hours had elapsed based on the patch application time, the patch was removed, and approximately 30 minutes later, the patch area was observed using a magnifying glass (MicroView) to confirm the presence or absence 5 of erythema and edema. Evaluation was conducted based on criteria in Table 12 below.

TABLE 12

| | | Skin | |
|---|---|---|---|
| Symbol | Score | Reaction | Evaluation Criteria |
| − | 0 | No | No reaction |
| | | irritation | |
| ± | 0.5 | Little | Faint or minimal erythema |
| | | irritation | |
| + | 1 | Mild | Erythema with clear |
| | | irritation | boundaries but mild erythema |
| ++ | 2 | Moderate | Marked erythema |
| | | irritation | |
| +++ | 3 | Strong | Severe erythema and blisters |
| | | irritation | |

Based on scores in Table 12 above, an average skin reactivity was calculated according to the formula below. The skin reaction results were calculated according to the formula below as a value proportional to a degree of irritation based on 100% for 3 points with the strongest skin irritation. In this case, the number of reactions is the number of subjects corresponding to each score.

$$Average skin reactivity = [ \frac{\sum ( Score \times number of reactions )}{Maximum score \times total number of subjects} ] \times 100$$

The results are shown in Table 13 below.

TABLE 13

| | Average |
|---|---|
| | skin |
| | reactivity |
| Comparative | 12.5 |
| Example 7 | |
| Example 4 | 3.3 |

As shown in the results in Table 13, a group treated with Example 4 was found to have an excellent skin soothing effect by improving skin reactivity by 370% or more compared to Comparative Example 7.

### INDUSTRIAL APPLICABILITY

A placenta-mimic cosmetic composition according to the present invention can show excellent skin improvement effects such as whitening, elasticity improvement, wrinkle improvement, moisturizing, or skin soothing by adding hydroxyethylpiperazine ethane sulfonic acid (HEPES) to polydeoxyribonucleotide (PDRN), which is an active ingredient in the placenta, and adding peptides, amino acids, or vitamins to recombine.

1. A placenta-mimic composition comprising polydeoxyribonucleotide (PDRN), and hydroxyethylpiperazine ethane sulfonic acid (HEPES).

2. The placenta-mimic composition of claim 1, further comprising one or more selected from the group consisting of peptides, amino acids, and vitamins.

3. The placenta-mimic composition of claim 2, wherein one or more combinations selected from the group consisting of yeast polypeptides, oligopeptide-1, oligopeptide-2, copper tripeptide-1, and acetyl hexapeptide-8 are used as the peptides.

4. The placenta-mimic composition of claim 2, wherein one or more selected from the group consisting of arginine, tryptophan, threonine, lysine, valine, leucine, phenylalanine, histidine, isoleucine, tyrosine, potassium aspartate, sodium glutamate, glutamine, asparagine, proline, alanine, glycine, and serine are used as the amino acids.

5. The placenta-mimic composition of claim 2, wherein one or more selected from the group consisting of biotin, folic acid, cyanocobalamin, niacinamide, pantothenic acid, pyridoxine, riboflavin, and thiamine are used as the vitamins.

6. A cosmetic composition comprising the placenta-mimic composition of claim 1.

7. The cosmetic composition of claim 6, wherein the placenta-mimic cosmetic composition is for skin improvement.

8. The cosmetic composition of claim 7, wherein the skin improvement is whitening, elasticity improvement, wrinkle improvement, moisturizing, or skin soothing.

9. A cosmetic composition comprising polydeoxyribonucleotide (PDRN), hydroxyethylpiperazine ethane sulfonic acid (HEPES), peptides, amino acids, and vitamins.

10. The cosmetic composition of claim 9, wherein one or more combinations selected from the group consisting of yeast polypeptides, oligopeptide-1, oligopeptide-2, copper tripeptide-1, and acetyl hexapeptide-8 are used as the peptides.

11. The cosmetic composition of claim 9, wherein one or more selected from the group consisting of arginine, tryptophan, threonine, lysine, valine, leucine, phenylalanine, histidine, isoleucine, tyrosine, potassium aspartate, sodium glutamate, glutamine, asparagine, proline, alanine, glycine, and serine are used as the amino acids.

12. The cosmetic composition of claim 9, wherein one or more selected from the group consisting of biotin, folic acid, cyanocobalamin, niacinamide, pantothenic acid, pyridoxine, riboflavin, and thiamine are used as the vitamins.

13. A quasi-drug composition comprising the placenta-mimic composition of claim 1.
